(19) United States
(12) Patent Application Publication
(10) Pub. No.: US 2020/0113469 A1
(43) Pub. Date: Apr. 16, 2020

(54) ELECTRODE ARRAY

(71) Applicant: CASE WESTERN RESERVE UNIVERSITY, Cleveland, OH (US)

(72) Inventors: Jayakumar Sahadevan, Beachwood, OH (US); Seungyup Lee, Cleveland, OH (US); Celeen Khrestian, Richmond Heights, OH (US); Albert Leon Waldo, Cleveland Heights, OH (US)

(21) Appl. No.: 16/158,961

(22) Filed: Oct. 12, 2018

(51) Int. Cl. A61B 5/042; A61B 18/14; A61B 5/00

(52) U.S. Cl. CPC A61B 5/0422; A61B 18/1477; A61B 2018/00839; A61B 18/1492; A61B 5/6852

(57) ABSTRACT

In an example, an electrode array for selective electrical sensing in patient tissue is described. The electrode array comprises an elongate base and a plurality of elongate fingers. Each finger is attached to and extends longitudinally from a distal end of the base. When at least a portion of the electrode array is in a spatulate use configuration, each finger terminates in a free end spaced longitudinally from the base and spaced laterally apart from adjacent free ends of other fingers. A plurality of electrode pairs is distributed along a contact surface of each finger. Respective electrodes in each of the electrode pairs of each finger are spaced from one another. The electrode pairs of each finger are both longitudinally and laterally spaced from electrode pairs of adjacent fingers when in the spatulate use configuration. An example method of selectively sensing electrical activity at a target body tissue is described.

### GOVERNMENT SUPPORT

This invention was made with government support under the grant(s) HL074189 awarded by the National Institutes of Health. The United States Government has certain rights in the invention.

### TECHNICAL FIELD

This disclosure relates to an apparatus and method for use of an electrode array.

### BACKGROUND

Electrical mapping of the heart is a procedure that is used to diagnose the origins of arrhythmias or other cardiac electrical characteristics. This procedure uses an electrically sensitive catheter to map the electrical activity of the heart.

As an example, to begin an electrical mapping procedure, a catheter sheath is inserted into a small incision in the arm or upper thigh. This process is usually visualized using x-rays, potentially in combination with a special dye that helps reveal the arteries (called angiography). This catheter is guided through the blood vessels until it is inside the heart. A smaller electrically sensitive catheter is then inserted inside the sheath and into the heart. This catheter can be used to sense electrical activity which may be mapped on a 3D model of the heart. The physician can use this mapping to understand electrical and/or mechanical function of the heart as well as to guide the performance of procedures such as ablation and cardiac resynchronization therapy.

### SUMMARY

In an example, an electrode array for selective electrical sensing in patient tissue is described. The electrode array comprises an elongate base and a plurality of elongate fingers. Each finger is attached to and extends longitudinally from a distal end of the base. When at least a portion of the electrode array is in a spatulate use configuration, each finger terminates in a free end spaced longitudinally from the base and spaced laterally apart from adjacent free ends of other fingers. A plurality of electrode pairs is distributed along a contact surface of each finger. Respective electrodes in each of the electrode pairs of each finger are spaced from one another. The electrode pairs of each finger are both longitudinally and laterally spaced from electrode pairs of adjacent fingers when in the spatulate use configuration.

In an example, a method of selectively sensing electrical activity at a target body tissue is described. The method includes providing an electrode array including an elongate base and a plurality of elongate fingers. Each finger is attached to and extends longitudinally from a distal end of the base. When at least a portion of the electrode array is in a spatulate use configuration, each finger terminates in a free end spaced longitudinally from the base and spaced laterally apart from adjacent free ends of other fingers. A plurality of electrode pairs is distributed along a contact surface of each finger. Respective electrodes in each of the electrode pairs of each finger are spaced from one another. The electrode pairs of each finger are both longitudinally and laterally spaced from electrode pairs of adjacent fingers when in the spatulate use configuration. The electrode array is placed into a compact bundle configuration. With the electrode array maintained in the compact bundle configuration, the electrode array is inserted into a vasculature of a patient and the electrode array is advanced to a use position adjacent the target body tissue. The electrode array is expanded from the compact bundle configuration to the spatulate use configuration. With the electrode array in the spatulate use configuration, electrical activity is sensed with the at least one selected electrode pair.

### BRIEF DESCRIPTION OF THE DRAWINGS

FIG. 1 is a top perspective view of an example electrode array.

FIG. 2 is a partial bottom view of the example electrode array of FIG. 1 in a first configuration.

FIG. 3 is a partial side view of the example electrode array of FIG. 1 in the second configuration.

FIG. 4 is a partial bottom view of the example electrode array of FIG. 1 in a second configuration.

FIG. 4A is a cross-sectional view taken along line A-A in FIG. 4.

FIG. 5 is a partial bottom view of the example electrode array of FIG. 1 in the third configuration.

FIG. 6 is a partial side view of the example electrode array of FIG. 1.

FIGS. 7-10 schematically depict example configurations of a component of the example electrode array of FIG. 1.

FIG. 11 is a flowchart of an example method of using the example electrode array of FIG. 1.

FIGS. 12-15 schematically depict an example use sequence of the example electrode array of FIG. 1.

### DETAILED DESCRIPTION

This disclosure relates to an apparatus and method for use of an electrode array. The electrode array includes a plurality of fingers, each of which carries spaced pairs of electrodes. The electrode array can be transformed between a compact bundle configuration—suitable for passing into a patient's body—and a spatulate use configuration. When in the spatulate use configuration, the fingers are splayed into a position to hold the electrodes in a predetermined pattern. The electrodes can be used to sense electrical activity in a target patient tissue. The pattern is useful in correlating the sensed electrical activity with particular areas of the target patient tissue.

The invention comprises, consists of, or consists essentially of the following features, in any combination.

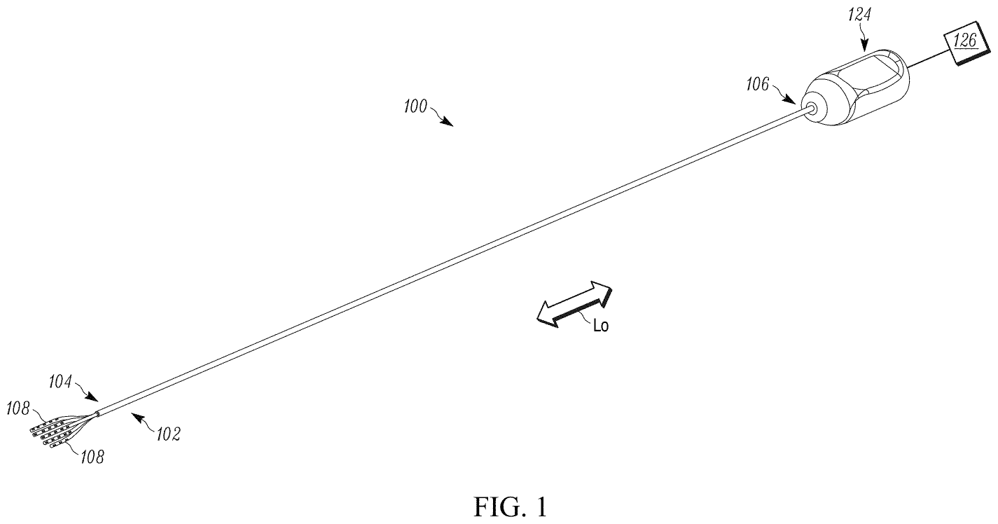

FIG. 1 depicts an example of an electrode array 100 for selective electrical sensing in patient tissue. The electrode array 100 includes an elongate base 102 having longitudinally spaced distal and proximal ends (104 and 106, respectively). The “longitudinal” direction, as used herein, is substantially parallel to arrow “Lo” in FIG. 1.

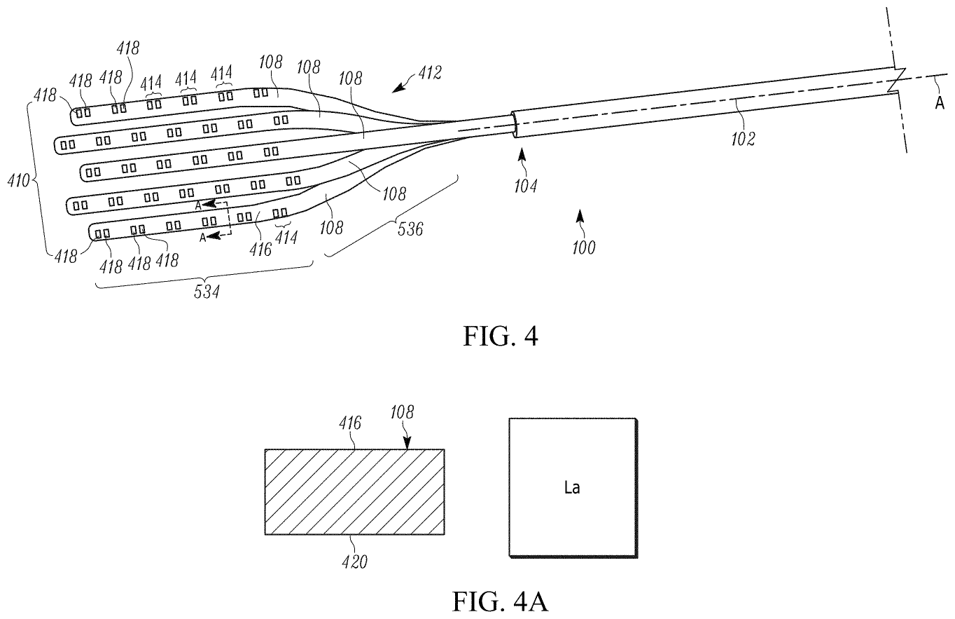

The electrode array 100 also includes a plurality of elongate fingers 108. Five fingers are shown in FIG. 1, but an electrode array 100 could include any desired number of fingers for a particular use environment. Each finger 108 is attached to and extends longitudinally from a distal end 104 of the base 102. As shown in FIG. 4A, at least a portion of each finger is rectangular in cross-section.

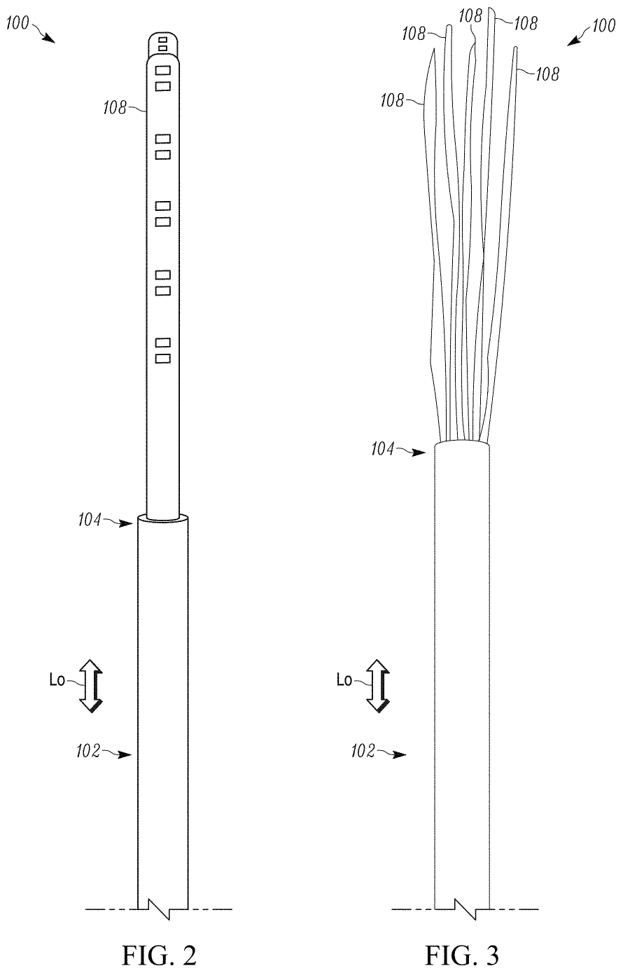

The electrode array 100 is selectively and collectively movable between a compact bundle configuration, in which the plurality of fingers 108 are gathered together to fit within a catheter body, and a spatulate use configuration, in which the plurality of fingers 108 are splayed into an expanded arrangement for deployment to sense electrical activity in patient tissue. The electrode array 100 is shown in bottom and side views, respectively, in an example of the compact bundle configuration in FIGS. 2-3. The electrode array 100 is shown in bottom view in the spatulate use configuration in FIG. 4.

The electrode array 100 may be moved between the compact bundle configuration and the spatulate use configuration in any desired manner including, but not limited to, the use of shape memory materials, mechanically biased finger 108 configurations, pressure exerted on the fingers 108 via a covering sheath or pull wire, any other suitable bundling or releasing mechanism or technology, or any combination thereof.

As is apparent from the view of FIG. 4, when at least a portion of the electrode array 100 is in the spatulate use configuration, each finger 108 terminates in a free end 410 spaced longitudinally from the base 102 and spaced laterally apart from adjacent free ends 410 of other fingers 108. For many use environments of the electrode array 100, the plurality of fingers 108 may be arranged in a reflectionally symmetric array about an axis of symmetry (“A” in FIG. 4) which is substantially coaxial with the base 102. The plurality of fingers 108 can be said to collectively form a spatulate electrode unit 412, as shown in at least FIG. 4, when in the spatulate use configuration.

A plurality of electrode pairs 414 is distributed along a contact surface 416 of each finger 108. Respective electrodes 418 in each of the electrode pairs 414 of each finger 108 are spaced from one another. The electrode pairs 414 of each finger 108 are both longitudinally and laterally spaced from electrode pairs 414 of adjacent fingers 108 when the electrode array 100 is in the spatulate use configuration. In the example electrode array 100 shown in the Figures, the electrodes 418 are on one surface of the fingers 108 with an opposing surface being free from electrode 418 or even electrically insulated, but it is contemplated that electrodes 418 could be present on any desired number of surface(s) of the fingers 108, and in any desired configuration(s).

As shown in FIG. 4, the electrode pairs 414 of each finger 108 may be longitudinally spaced from one another along the length of each respective finger 108. The electrode pairs 414 may be located only on the contact surface 416 of each finger, and a laterally opposed second surface 420 of each finger 108 may be electrically insulated. The term “lateral”, as used herein, indicates a direction substantially perpendicular to the longitudinal direction. As represented by plane La, the “lateral” direction is substantially coincident with the page in FIG. 4A. Each electrode 418, as shown in the Figures, may have a rectangular footprint and be substantially planar along the contact surface 416 (i.e., substantially parallel to the contact surface 416) of the respective finger 108 upon which that electrode 414 is carried.

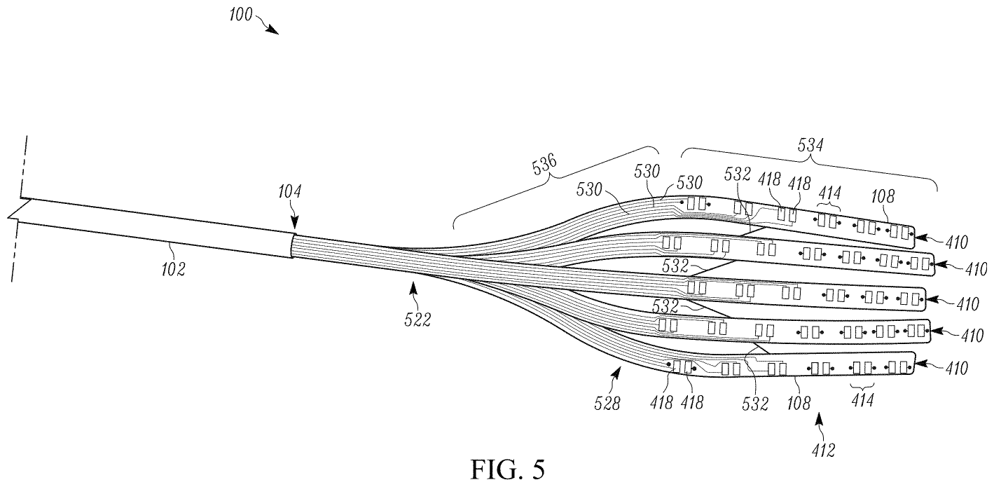

With reference now to FIG. 5, the electrode array 100 may include conductive line 522 placing each of the electrodes 418 into electrical communication with the proximal end 106 of the base 102. The electrode array 100 may include a handle 124 (shown in FIG. 1), to provide a user with a graspable control surface. The array further may include a connection interface, such as residing on or attached to the handle 124 to provide a physical layer (e.g., wired and/or wireless) connection between the electrode array 100 and an external device 126 (shown schematically). The connection interface thus can provide for power transmission, signal transmission, or for any other desired reason between the external device 126 and the electrode array 100. For example, the external device 126 can include signal processing circuitry (e.g., filters and amplifiers, analog-to-digital converters and the like) to convert the sensed electrical signals into digital dipole signal data for each electrode pair 414. A computer (e.g., including one or more processor and memory) further can execute instructions to further process the digital dipole signal data, such as to map the sensed dipole signals onto a model of the heart.

While the present description references dipole signal data, it should be understood that, for certain use embodiments, each electrode's 418 data may be acquired individually. The unipolar data from multiple electrodes 418 can then be combined to form a dipolar signal for an electrode pair 414, or even a higher order signal for more electrodes. However, the unipolar data from each individual electrode 418 can be saved for later review as desired. It is also contemplated that the unipolar data itself can be used in the analysis, in lieu of the referenced dipolar data, for certain use environments of the electrode array 100.

As shown in the example of FIG. 5, at least one finger 108 may comprise a flex circuit strip 528 including electrically conductive traces 530 (serving as the aforementioned conductive line 522) extending from each electrode 418 along the length of the respective finger 108 and to the base 102 (e.g., terminating at the connection interface). For example, the trace may be formed of a metal (e.g., copper, aluminum, sliver or gold), a conductive polymer and/or a conductive ink disposed on the flex circuit strip. The electrode can be formed of the same or different material. When each finger is comprised of a flex circuit strip 528, at least two of the fingers 108 may be stacked together into a laminated stack of flex circuit strips 528 when the electrode array 100 is in the compact bundle configuration shown in FIGS. 2-3.

It is also contemplated that others of the fingers 108 could concurrently rotate during the transition into the compact bundle configuration. In this way, at least one of the fingers 108 could be oriented differently from others of the fingers 108 when collapsed into the compact bundle configuration (e.g., for insertion into a body lumen), but could reorient (e.g., rotate in the lateral plane) into a position substantially laterally aligned with the other fingers 108 when in the spatulate use configuration.

At least one flexible connecting element 532, as shown in FIG. 5, may connect adjacent fingers 108 to limit lateral separation between the fingers 108 when the electrode array 100 is in the spatulate use configuration. That is, the flexible connecting elements 532 could act in a “webbing” type manner to restrain adjacent fingers 108 from splaying further apart than a predetermined finger separation distance and thereby maintain a desired spatial arrangement of the fingers and associated electrode pairs. Since proper electrode 418 placement can facilitate accurate cardiac mapping, known and repeatable deployment of the fingers 108 from compact bundle configuration to spatulate use configuration may be desirable. Similarly, one or more rigid connecting element(s) (not shown) could be provided to operate like an umbrella rib, to urge the fingers 108 toward the spatulate use configuration.

In some examples, at least one finger 108 may include a shape-memory substrate material along a longitudinal portion thereof, to which the flex circuit strip 528 is attached to at least partially form the finger 108. When present, the shape-memory substrate material operates (e.g., via one-way or two-way shape memory effect) to urge the electrode array from the compact bundle configuration to the spatulate use configuration, such as when such transformation takes place within the patient's body, near the target body tissue. For example, the spatulate use configuration can be the original shape of the shape memory material for the electrode array 100. A user can cool the electrode array 100, including one-way shape memory substrate material, and manipulate the array into the compact bundle configuration. Alternatively, for two-way shape memory material, cooling can cause the electrode array 100 to automatically transform to the compact bundle configuration. When positioned within the body, the electrode array 100 can heat to (or above) the transition temperature to cause the electrode array 100 to expand to its spatulate use configuration.

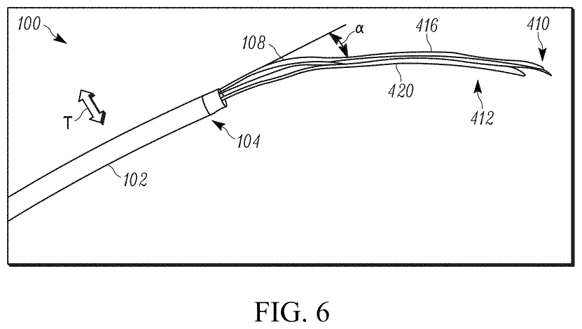

As shown in the example of FIG. 6, the spatulate electrode unit 412 may have a convex configuration in the transverse direction. The term “transverse” is used here to indicate a special case of a lateral direction, and the transverse direction is represented by arrow T in FIG. 6. Also or instead of having a convex curvature, the spatulate electrode unit 412 may extend longitudinally at a transverse wrist angle (shown as a in FIG. 6) from the base 102. The convex configuration and/or transverse wrist angle of the spatulate electrode unit 412 can be provided for a particular use environment.

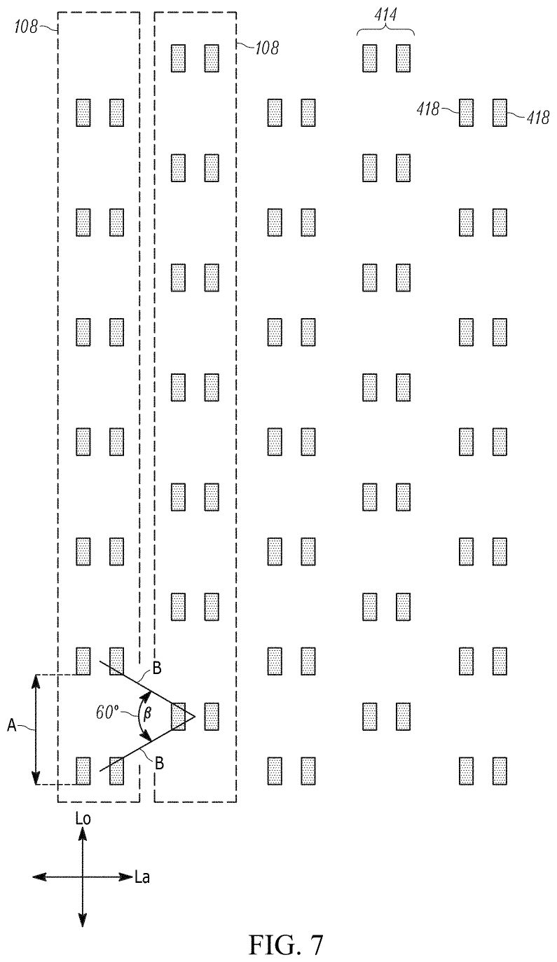

In the spatulate use configuration, the plurality of fingers 108 may extend substantially parallel to one another along at least an electrode-containing portion 534 of the length thereof, as shown in FIG. 4. Accordingly, the electrode-containing portions 534 of the plurality of fingers 108 can help to spatially arrange the electrode pairs 414 carried by those fingers 108 into a grid-type pattern, such as shown in FIGS. 7-10 and discussed further below. It should be noted, though, that, when the electrode-containing portions 534 of the fingers 108 are arranged in the parallel arrangement shown in FIG. 4, at least one finger 108 may further include, as a portion of its length, a non-electrode-containing portion 536 that extends angularly between the base 102 and the electrode-containing portion 534 of the length of the at least one finger 108 when in the spatulate use configuration. Stated differently, at least one finger 108 could include an electrode-containing portion 534 and a non-electrode-containing portion 536 along its length, and the non-electrode-containing portion 536 extends longitudinally from the base 102 at an angle, such that the corresponding electrode-containing portion 534 is cantilevered longitudinally out from the base 102 at a particular lateral spacing with respect to the other fingers 108.

Turning now to FIGS. 7-10, several example electrode 418 arrangements and patterns are shown in detail, though one of ordinary skill in the art could readily configure an electrode array 100 having a desired layout for a particular use environment. In FIGS. 7-10, the fingers 108 and other electrode array 100 components are largely omitted for clarity (only shown schematically in phantom line in part of FIG. 7), but one of ordinary skill in the art will understand that each column of electrodes 418 shown represents the pattern and spacing of electrodes 418 corresponding to a finger 108, such that the “grid” pattern of each of FIGS. 7-10 represents the composite overall pattern of electrodes 418 corresponding to a particular spatulate electrode unit 412. However, FIGS. 7-10, like all Figures in this application, are not shown as being to scale unless explicitly noted as such.

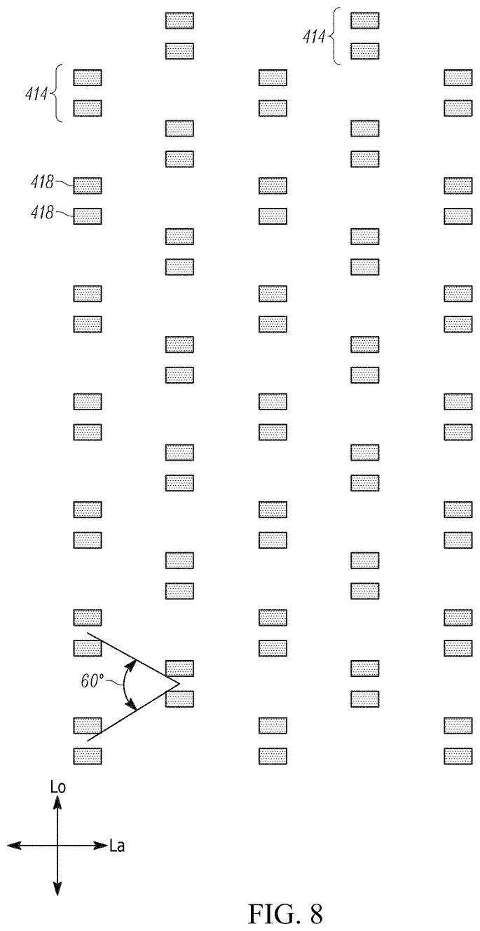

As shown in FIG. 7, respective electrodes 418 in each of the electrode pairs 414 of each finger 108 may be laterally spaced from (left-right in this view), and longitudinally aligned with (top-bottom in this view), one another. In contrast, as shown in FIGS. 8-10, respective electrodes 418 in each of the electrode pairs 414 of each finger 108 may instead be longitudinally spaced from (top-bottom in this view), and laterally aligned with (left-right in this view), one another.

The electrodes 418 may be arranged in electrode pairs 414 with a specified distance between individual electrodes of a pair 414 (e.g., about 1.5 mm between centers of electrodes in the pair). Each electrode pair 414 has a “center-pair” point which is located between the two electrodes 418, and equidistant from both. For sake of consistency, the “center-pair” point is referenced below.

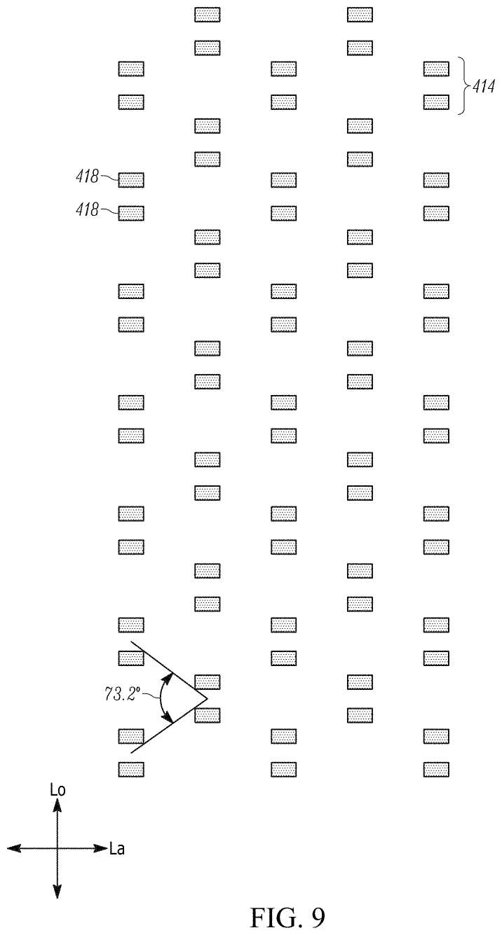

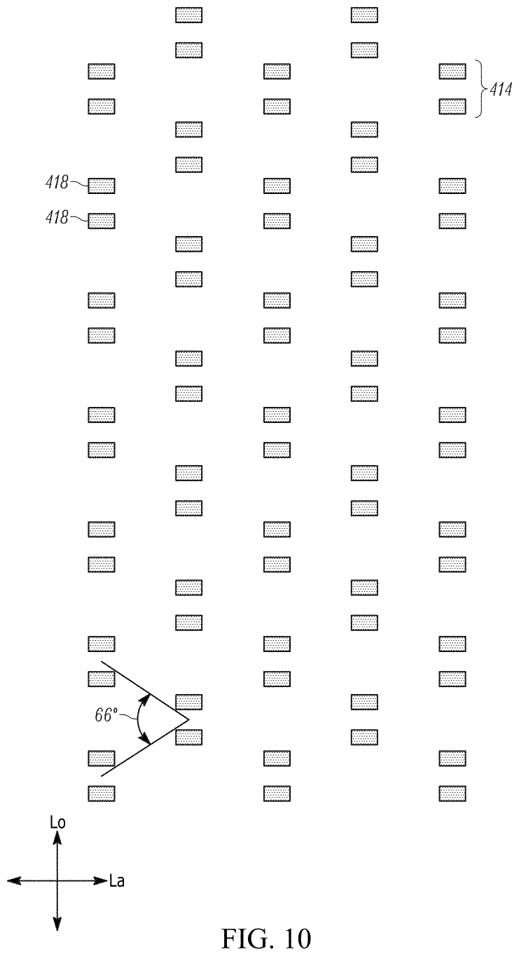

Adjacent electrode pairs 418 of a selected finger 108 may have a longitudinal center-pair-to-center-pair spacing in the range of, for example, 5.0-5.5 mm. An example of this distance is shown at “A” in FIG. 7. Adjacent electrode pairs 418 of two different adjacent fingers 108 may have a diagonally lateral center-pair-to-center-pair spacing in the range of, for example, 4.3-5.3 mm. An example of this distance is shown at “B” in FIG. 7. Adjacent electrode pairs 418 of two different adjacent fingers 108 may have a diagonally lateral orientation angle (shown at “β” in FIG. 7) in the range of approximately 45°-75°. By way of example, the diagonally lateral orientation angle β is about 60° in FIGS. 7-8, about 73° in FIG. 9, and about 66° in FIG. 10. In other examples, the actual spacing and layout of the electrode pairs 418 may differ slightly in practice from the predetermined layouts (such as in FIGS. 7-10), but one of ordinary skill in the art will be able to use mechanical and/or software error correction to bring any spatial differences into acceptable conformance with desired accuracy ranges, in practice.

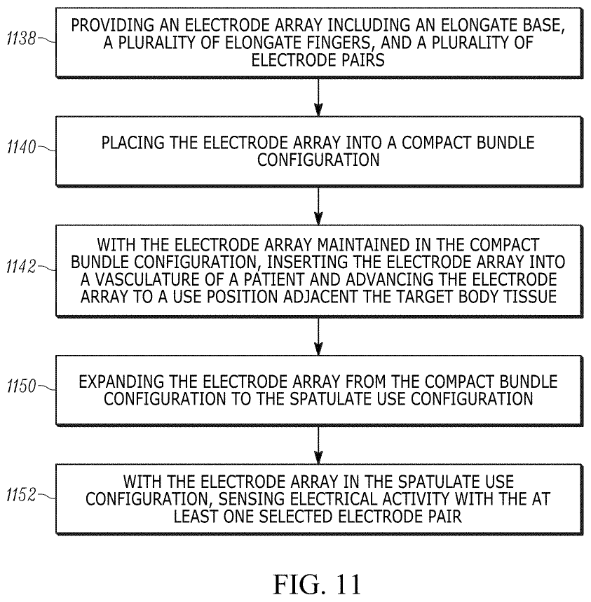

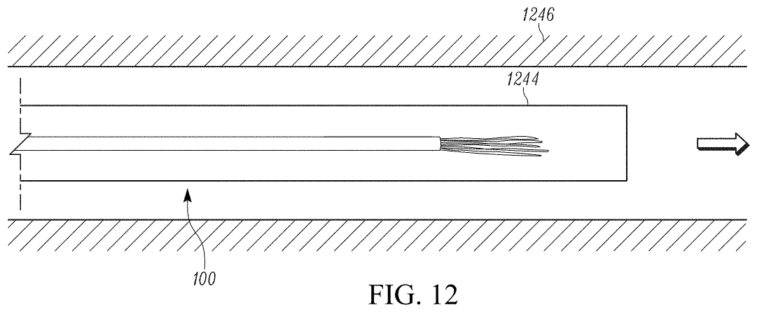

FIG. 11 is a flowchart schematically depicting the example method of using the electrode array 100, which is also shown pictorially in FIGS. 12-15. In FIG. 11, a method of selectively sensing electrical activity at a target body tissue is outlined. In first action block 1138, an electrode array 100 is provided. The electrode array 100 includes an elongate base 102, a plurality of elongate fingers 108, and a plurality of electrode pairs 414. Each finger 108 is attached to and extends longitudinally from a distal end 104 of the base 102. When at least a portion of the electrode array 100 is in a spatulate use configuration, each finger 108 terminates in a free end 410 spaced longitudinally from the base 102 and spaced laterally apart from adjacent free ends 410 of other fingers 108. The plurality of electrode pairs 414 is distributed along a contact surface 416 of each finger 108 while, in some examples, the surface opposite the contact surface is free of electrodes 418. Respective electrodes 418 in each of the electrode pairs 414 of each finger 108 are spaced from one another. The electrode pairs 414 of each finger 108 may be longitudinally and/or laterally spaced from electrode pairs 414 of adjacent fingers 108 when the electrode array 100 is in the spatulate use configuration.

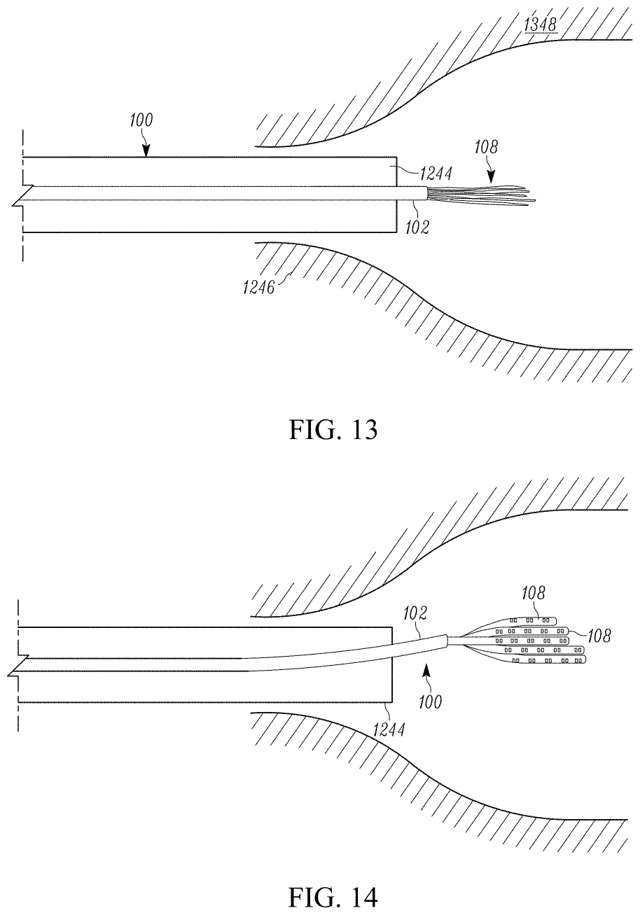

The method then proceeds to second action block 1140, where the electrode array 100 is placed into a compact bundle configuration (as shown in FIGS. 2-3). As shown in FIG. 12 and described in third action block 1142, the electrode array 100 is maintained in the compact bundle configuration (such as by being constrained within sheath 1244 and/or maintained by material properties of a substrate material), and is inserted into a vasculature 1246 of a patient. The electrode array 100 is advanced to a use position adjacent the target body tissue 1348, as shown in FIG. 13, which may be done with the assistance of sheath 1244.

Once the electrode array 100 is located adjacent the target body tissue 1348 as desired, as shown in FIG. 13, the electrode array 100 may be expanded from from the compact bundle configuration to the spatulate use configuration in fourth action block 1150, and as shown in the transition from FIG. 13 to FIG. 14. For example, the expansion from the compact bundle configuration to the spatulate use configuration may occur automatically and/or in response to user actuation. It is noted that these Figures are merely schematic and that the electrode array 100 does not necessarily rotate about the axis A of the base as shown in these Figures; these depictions are included with the understanding that the side view of the compact bundle configuration of the electrode array 100 is similar to the side view of the spatulate use configuration. It is also noted that, for self-expanding versions of the electrode array 100, the compact bundle configuration could be urged into that configuration and/or restricted by sheath 1244 or another constraining structure (not shown). As a result, in such an arrangement, the electrode array 100 would not be expected to be in the compact bundle configuration without the influence of a constraining structure surrounding the electrode array 100 to prevent self-expansion into the spatulate use configuration.

The electrode array 100 may be expanded as in the fourth action block 1150 in any desired manner. For example, when sheath 1244 is present, the electrode array 100 could be manipulated to extend from the distal end of the sheath 1244 and/or self-expand due to shape-memory material. Lateral separation between the fingers 108 may be limited via at least one flexible connecting element 532 connecting adjacent fingers 108, as previously described. Also as previously described, the plurality of fingers 108 could collectively form a spatulate electrode unit 412 having a convex configuration in the transverse direction.

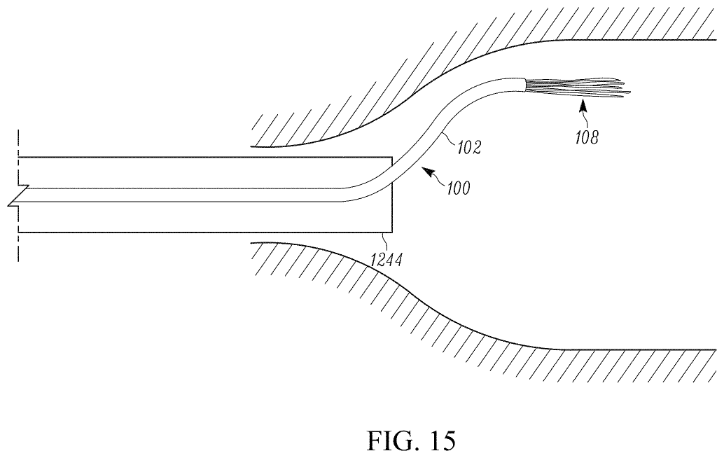

Regardless of the way in which the electrode array 100 reaches the spatulate use configuration, though, once such use configuration has been achieved, the electrical activity of the target body tissue 1348 may be sensed. For example, electrodes 418 of one or more electrode pair 414 may sense electrical activity at the target body tissue, as shown in FIG. 15 and described in fifth action block 1152 of FIG. 11. This could be accomplished via contact between one or more electrode(s) 418 and the target body tissue, or at least partially through the use of non-contact sensing. In some examples, the target body tissue includes endocardial tissue. In other examples, the target body tissue includes epicardial tissue. For instance, once desired sensing has been performed at the target body tissue, the electrode array 100 may be moved to another target location. In order to communicate the sensed electrical activity, the electrode pairs 414 may be placed into electrical communication with the proximal end 106 of the base 102 via the conductive line 522 (e.g., electrical traces). In such case, sensing electrical activity with the at least one selected electrode pair 414 will include communicating the sensed electrical signals from the at least one selected electrode pair 414, through the conductive line 522, to the base 102, which further may be communicated to an external device (e.g., processing circuitry and computer) via a corresponding interface.

Beginning with the electrode array 100 in a use position adjacent the target body tissue 1348, once the electrical sensing is complete as desired, the electrode array 100 may be collapsed from the spatulate use configuration to the compact bundle configuration. Then, with the electrode array 100 maintained in the compact bundle configuration, the electrode array 100 may be withdrawn from the vasculature 1246 of the patient. The data collected from the electrical sensing facilitated by the electrode array 100 can be used to perform cardiac mapping that can then be employed to guide ablative or other procedures. It is contemplated that the electrode array 100, or portions thereof, could be used to apply electrical signals to the target patient tissue 1348 in particular use environments.

What have been described above are examples. It is, of course, not possible to describe every conceivable combination of structural and functional features or methodologies, but one of ordinary skill in the art will recognize that many further combinations and permutations are possible.

Unless defined otherwise, all technical and scientific terms used herein have the same meaning as is commonly understood by one of skill in the art to which the present disclosure pertains.

As used herein, the term “and/or” can include any and all combinations of one or more of the associated listed items.

As used herein, phrases such as “between X and Y” and “between about X and Y” can be interpreted to include X and Y.

It will be understood that when an element is referred to as being “on,” “attached” to, “connected” to, “coupled” with, “contacting,” etc., another element, it can be directly on, attached to, connected to, coupled with or contacting the other element or intervening elements may also be present

As used herein, the phrase “at least one of X and Y” can be interpreted to include X, Y, or a combination of X and Y. For example, if an element is described as having at least one of X and Y, the element may, at a particular time, include X, Y, or a combination of X and Y, the selection of which could vary from time to time. In contrast, the phrase “at least one of X” can be interpreted to include one or more Xs.

As used herein, the term “includes” means includes but not limited to, the term “including” means including but not limited to. Additionally, where the disclosure or claims recite “a,” “an,” “a first,” or “another” element, or the equivalent thereof, it should be interpreted to include one or more than one such element, neither requiring nor excluding two or more such elements.

While aspects of this disclosure have been particularly shown and described with reference to the example aspects above, it will be understood by those of ordinary skill in the art that various additional aspects may be contemplated. For example, the specific methods described above for using the apparatus are merely illustrative; one of ordinary skill in the art could readily determine any number of tools, sequences of steps, or other means/options for placing the above-described apparatus, or components thereof, into positions substantively similar to those shown and described herein. In an effort to maintain clarity in the Figures, certain ones of duplicative components shown have not been specifically numbered, but one of ordinary skill in the art will realize, based upon the components that were numbered, the element numbers which should be associated with the unnumbered components; no differentiation between similar components is intended or implied solely by the presence or absence of an element number in the Figures. Any of the described structures and components could be integrally formed as a single unitary or monolithic piece or made up of separate sub-components, with either of these formations involving any suitable stock or bespoke components and/or any suitable material or combinations of materials; however, the chosen material(s) should be biocompatible for many applications. Any of the described structures and components could be disposable or reusable as desired for a particular use environment. A “predetermined” status may be determined at any time before the structures being manipulated actually reach that status, the “predetermination” being made as late as immediately before the structure achieves the predetermined status.

The term “substantially” is used herein to indicate a quality that is largely, but not necessarily wholly, that which is specified, allowing some amount of variation. Though certain components described herein are shown as having specific geometric shapes, all structures of this disclosure may have any suitable shapes, sizes, configurations, relative relationships, cross-sectional areas, or any other physical characteristics as desirable for a particular application. Any structures or features described with reference to one example or configuration could be provided, singly or in combination with other structures or features, to any other aspect or configuration, as it would be impractical to describe each of the aspects and configurations discussed herein as having all of the options discussed with respect to all of the other aspects and configurations. A device or method incorporating any of these features should be understood to fall under the scope of this application, including the appended claims.

Accordingly, the disclosure is intended to embrace all such alterations, modifications, and variations that fall within the scope of this application, including the appended claims.

What is claimed is:

1. An electrode array for selective electrical sensing in patient tissue, the electrode array comprising:
an elongate base;
a plurality of elongate fingers, each finger being attached to and extending longitudinally from a distal end of the base and, when at least a portion of the electrode array is in a spatulate use configuration, each finger terminates in a free end spaced longitudinally from the base and spaced laterally apart from adjacent free ends of other fingers; and
a plurality of electrode pairs distributed along a contact surface of each finger, respective electrodes in each of the electrode pairs of each finger being spaced from one another and the electrode pairs of each finger being both longitudinally and laterally spaced from electrode pairs of adjacent fingers when in the spatulate use configuration.

2. The electrode array of claim 1, wherein respective electrodes in each of the electrode pairs of each finger are longitudinally spaced from, and laterally aligned with, one another.

3. The electrode array of claim 1, wherein respective electrodes in each of the electrode pairs of each finger are laterally spaced from, and longitudinally aligned with, one another.

4. The electrode array of claim 1, wherein the plurality of fingers extend substantially parallel to one another along at least an electrode-containing portion of the length thereof.

5. The electrode array of claim 1, including at least one flexible connecting element connecting adjacent fingers to limit lateral separation between the fingers when the electrode array is in the spatulate use configuration.

6. The electrode array of claim 1, wherein the plurality of fingers are arranged in a reflectionally symmetric array about an axis of symmetry coaxial with the base.

7. The electrode array of claim 1, wherein the plurality of fingers collectively form a spatulate electrode unit when in the spatulate use configuration.

8. The electrode array of claim 7, wherein the spatulate electrode unit has a convex configuration in the transverse direction.

9. The electrode array of claim 1, wherein the plurality of fingers are selectively and collectively movable between a compact bundle configuration, in which the plurality of fingers are gathered together to fit within a catheter body, and the spatulate use configuration.

10. The electrode array of claim 1, wherein each finger is comprised of a flex circuit strip, and at least two of the fingers are stacked together in a laminated stack of flex circuit strips when in a compact bundle configuration.

11. The electrode array of claim 9, wherein at least one finger comprises a shape-memory substrate material to which the flex circuit strip is attached.

12. The electrode array of claim 1, wherein adjacent electrode pairs of two different adjacent fingers have a diagonally lateral orientation angle in the range of 45°-75°.

13. The electrode array of claim 1, including conductive line placing the electrode pairs into electrical communication with a proximal end of the base.

14. The electrode array of claim 12, wherein at least one finger comprises a flex circuit strip including electrically conductive traces extending from each electrode along the length of the respective finger and to the base.

15. The electrode array of claim 1, wherein each electrode has a rectangular footprint and is substantially planar along the contact surface of the finger.

16. A method of selectively sensing electrical activity at a target body tissue, the method comprising:
providing an electrode array including
an elongate base,
a plurality of elongate fingers, each finger being attached to and extending longitudinally from a distal end of the base and, when at least a portion of the electrode array is in a spatulate use configuration, each finger terminates in a free end spaced longitudinally from the base and spaced laterally apart from adjacent free ends of other fingers, and
a plurality of electrode pairs distributed along a contact surface of each finger, respective electrodes in each of the electrode pairs of each finger being spaced from one another and the electrode pairs of each finger being both longitudinally and laterally spaced from electrode pairs of adjacent fingers when in the spatulate use configuration;
placing the electrode array into a compact bundle configuration;
with the electrode array maintained in the compact bundle configuration, inserting the electrode array into a vasculature of a patient and advancing the electrode array to a use position adjacent the target body tissue;
expanding the electrode array from the compact bundle configuration to the spatulate use configuration; and
with the electrode array in the spatulate use configuration, sensing electrical activity with the at least one selected electrode pair.

17. The method of claim 16, including:
beginning with the electrode array in a use position adjacent the target body tissue, collapsing the electrode array from the spatulate use configuration to the compact bundle configuration; and
with the electrode array maintained in the compact bundle configuration, withdrawing the electrode array from the vasculature of the patient.

18. The method of claim 16, including, when the electrode array is in the spatulate use configuration, limiting lateral separation between the fingers via at least one flexible connecting element connecting adjacent fingers.

19. The method of claim 16, wherein expanding the electrode array from the compact bundle configuration to the spatulate use configuration includes, with the plurality of fingers, collectively forming a spatulate electrode unit having a convex configuration in the transverse direction.

20. The method of claim 16, including placing the electrode pairs into electrical communication with a proximal end of the base via conductive line; and
sensing electrical activity with the at least one selected electrode pair includes transmitting electrical signals from the at least one selected electrode pair, through the conductive line, to the base.
